(19) United States
(12) Patent Application Publication
(10) Pub. No.: US 2021/0138303 A1
(43) Pub. Date: May 13, 2021

(54) SYSTEM AND METHOD FOR MEASURING A SPORTS PERFORMANCE AND DISPLAYING THE RESULTS

(71) Applicant: ANGULAR VELOCITY OY, Helsinki (FI)

(72) Inventors: Kristian TÖRNQVIST, Vantaa (FI); Juha MAJA, Kuortane (FI); Or AVIRAM, Tel Aviv (IL); Önder GURKAN, Ankara (TR); Patrick ELLIS, Helsinki (FI); Jukka PUHAKKA, Siuntio (FI)

(21) Appl. No.: 17/155,173

(22) Filed: Jan. 22, 2021

Related U.S. Application Data
(63) Continuation of application No. PCT/FI2019/050510, filed on Jun. 27, 2019.

(30) Foreign Application Priority Data
Jul. 4, 2018 (FI) 20185621

(51) Int. Cl. A63B 24/00

(52) U.S. Cl. CPC A63B 24/0062; A63B 24/0006; A63B 2024/0015; A63B 2225/20; A63B 2220/805; A63B 2220/806; A63B 2220/89; A63B 2220/10

(57) ABSTRACT

A method and system for monitoring sports performance of an athlete includes a sensor for monitoring movement of the athlete, a sensor for monitoring a position of the athlete, and a controller for receiving information of the monitored signals from the sensors, wherein the controller includes a filter for filtering the received signals. The system also includes a cloud database for receiving and storing the data sent by the controller, and the cloud includes a filter for filtering and processing the data received from the controller. The system further includes a user interface for displaying the processed data showing the sports performance, received from the cloud.

### FIELD OF THE DISCLOSURE

The disclosure relates to a method and a system for measuring a sports performance and displaying results and analysis of the performance to a user. The sports performance may be any performance performed by an athlete or other sportsman and may vary in duration and field.

### BACKGROUND OF THE DISCLOSURE

Traditionally sports performances have been analysed by filming the event itself and from a picture to picture analysing and monitoring the movement of the athlete as a whole or a single feature such as stride length or an angle of the knee or any other feature of interest. As the technique and the perfection of it are very important in many sports, this has not been advantageous as it requires a lot of manual work and analysing and is a slow process.

### BRIEF DESCRIPTION OF THE DISCLOSURE

An object of the present disclosure is to provide a system and method for monitoring and measuring a sports performance and displaying results where at least one of disadvantages of the prior art is eliminated or at least alleviated. The objects of the present invention are achieved with a system, method and computer program product according to the characterizing portions of the independent claims.

The present invention is directed to a method for monitoring a sports performance of an athlete comprising detecting in at least one sensor movement of an athlete, detecting in at least one sensor a position of an athlete, sending out the detected signals to a controller from the sensor, filtering the data constituting of the received signals in the controller, sending the filtered data to a cloud database, storing the data in the cloud database, filtering the data further by means for filtering in the cloud, sending out the filtered data, and displaying the data on a user interface for a user.

The invention is further directed to a system for monitoring a sports performance comprising means for carrying out said method.

The preferred embodiments of the invention are disclosed in the dependent claims.

### BRIEF DESCRIPTION OF THE DRAWINGS

In the following the disclosure will be described in greater detail by means of preferred embodiments with reference to the accompanying drawings, in which

FIG. 1 shows a flow chart of a process with a single sensor according to one embodiment of the invention.

FIG. 2 shows a flow chart of a process with multiple sensors according to another embodiment of the invention.

### DETAILED DESCRIPTION OF SOME EMBODIMENTS

The following embodiments are exemplary. Although the specification may refer to “an”, “one”, or “some” embodiment(s), this does not necessarily mean that each such reference is to the same embodiment(s), or that the feature only applies to a single embodiment. Single features of different embodiments may be combined to provide further embodiments.

In the following, features of the invention will be described with a simple example of a device architecture in which various embodiments of the invention may be implemented. Only elements relevant for illustrating the embodiments are described in detail.

The present invention is directed to a system for monitoring a sports performance of an athlete comprising a sensor for monitoring movement of the athlete, a sensor for monitoring a position of the athlete, a controller for receiving information of the monitored signals from the sensors, the controller comprising a filter for filtering the received signals, a cloud database for receiving and storing the data sent by the controller, the cloud comprising means for filtering and processing the data received from the controller, a user interface for displaying the processed data showing the sports performance, received from the cloud.

The invention is also directed to a method for monitoring a sports performance of an athlete comprising detecting in at least one sensor movement of an athlete, detecting in at least one sensor a position of an athlete, sending out the detected signals to a controller from the sensor, filtering the data constituting of the received signals in the controller, sending the filtered data to a cloud database, storing the data in the cloud database, filtering the data further by means for filtering in the cloud, sending out the filtered data, and displaying the data on a user interface for a user.

According to the invention the sensor or sensors are detecting signals and sending the unprocessed data, or raw data to a controller. The controller determines whether there is anything to measure or if the data is to be discarded. If the received data contains measurement data regarding movement, the controller will filter the data in a filter in order to filter out irrelevant signals and noise. The filtered data is then sent to a cloud database where it is filtered further and processed in order to be sent out to users.

If the received data does not contain any measurement data regarding movement, or if the controller determines that the data contains invalid sensor readings, e.g. anomalous position of the subject, or a so-called ghost subject, i.e. an event is detected where there is none, the controller will discard the data.

The processed data is then sent from the cloud database to the users and displayed via a user interface. The user interface may be a smartphone, a pad, a laptop or PC or may be displayed during a sports event on a big screen or during a TV broadcast.

The controller is adapted to send out control commands to a sensor or sensors. Possible control commands may be a change of frequency, rotational speed, show status or another needed command. The controller may also send out its own status information to the cloud database.

The controller is adapted to receive control commands from the cloud, such as reboot, report, system status start/stop and configuration information.

The cloud database will save data received from the controller. Further the data will be filtered in order to be sent to the users. The cloud may additionally push out finalized data to users who are online. Further the cloud may receive data from the users about the location of the client, which version of the user interface they are using and if it is working properly or not.

The user can via the interface see the data in real time, search for archived data and check their own data.

The data displayed to the user may be results of measurements, e.g. time, stride length, speed, step position, angles of e.g. a foot or leg, stride frequency and other data that might be of interest for the user. The user may be an athlete, a trainer, TV-viewers or other interested parties.

In more detail, the sensor will send out a continuous stream of measurement data, which data is then broken down into components, such as time angle, distance, reflection intensity and so on. with this information a so-called point cloud is generated. By a point cloud is here meant a set of data points in space. Point clouds are generally produced by 3D scanners, which measure a large number of points on the external surfaces of objects around them.

With this information it is determined what is detected, shape, direction, speed, recording angles to mention a few. This is then stored in a local database in the controller where it is determined whether it is relevant and thus saved or irrelevant and thus discarded.

If the data is determined to be relevant it is sent to a cloud database. In the database all measurement parameters are stored in tables. In the cloud the data is then linked to who did perform, where it was performed, when and how. Based on this a certain event can be searched by the user through the user interface.

The cloud database keeps track of which controllers and sensors are active and communicate. It also keeps track of what client apps e.g. tablets, smartphones etc. are connected and what data they are requesting to see. If a client is connected to view triple jump data for example, his access is verified and the type of data, to ensure that a user gets the right data and not data concerning some other sport.

The cloud also generates broader statistics for live views, average jump length, fastest run times etc.

All communication between the component of the system are encrypted to ensure correctness of the data.

A variety of sensors may be used in the system, some examples being a laser scanner, or a LiDAR. With a LiDAR step position, stride length, step frequency, contact time, speed, acceleration, deceleration, maximum speed, minimum speed, flight time to mention a few, may be measured.

Another example of a sensor that may be used in the system is a 3D camera comprising an IR depth mapper. The IR depth mapper makes a topographical map of the subject to be detected and thus created a 3D model of the subject and a normal video feed. With a 3D camera body and joint positions or angles may be measured.

According to one embodiment of the invention multiple sensors are used together and prioritized differently depending of the sport to be monitored and what information is the relevant one.

According to one embodiment of the invention the sensors are constantly measuring everything inside their measurement radius. The controller will process all the data where it is determined according to mathematical formulas or algorithms whether the data is to be used or discarded. The data that is to be saved, is processed again and it is then determined if the measured subject or object is for example a foot, a javelin or something else. According to the embodiment it is also possible to instruct the controller on what type of object that is of relevance at the time of measurement.

FIG. 1 shows a flow chart according to one embodiment of the invention. The sensor is detecting movement and sends the detected data or raw data to the controller. The controller may send sensor control commands to the sensor. The controller sends filtered data to the cloud, or discards the data coming from the sensor if it is determined to be irrelevant. The cloud may send control messages to the controller. The cloud sends out the data in its final form to be displayed to the user. Further the cloud may also receive data from the users.

FIG. 2 shows an alternative embodiment of the invention where multiple sensors are sending measurement data to the controller.

If the sport to be monitored is running, for example 100m, 200 m, 400 m and so on the most relevant sensor is a Lidar or a laser scanner. It measures step position, stride length, step frequency, speed, acceleration and deceleration. A 3D camera may provide additional information on body angles, joint positions and so on, which may be relevant in determining why one athlete is faster than another or is accelerating more powerfully.

In throwing disciplines such as discus, hammer, javelin or shot put the 3D camera is the most relevant sensor. Here the throwing angle, speed, body angles among others are of most interest. A LiDAR or laser scanner may be relevant if the approach run is of interest as in javelin. In this case stride length, step position and speed are at least of interest.

In jumping disciplines such as long jump or triple jump the approach run is measured with a LiDAR and the 3D camera is used for analyzing body angles.

In jumping disciplines such as high jump and pole vault a 3D camera is used for monitoring the jump itself, among others body angles, the total maximum height of the jump, the height of the gap between the bar and the highest body position, timing of the jump in relation to the bar, sideways position to name the most relevant. A LiDAR is again used to analyze the approach run.

The system according to the invention may also be used in biathlon for target monitoring and determining if a shot hits the target or not. An advantage of using the system according to the invention in biathlon is that a single sensor can monitor tens of targets and ranges at once. The system according to the invention is also more reliable and less prone to be affected by the weather and faster than a conventional system using IP cameras, in other words any type of cameras capable of sending image data over a network.

According to the invention the data, when it has been determined as valid data by the controller, is processed once more in order to determine if the object/subject detected is a foot or e.g. a javelin or a hammer. For example, in case of long jump it is analyzed based on the data where on the track a step was taken and with which foot, left or right, for how long the foot was at this position and when it disappeared. From this information speed, direction, flight time, contact time and other measurement values of the approach run and jump are calculated.

According to one embodiment several scans are conducted before the determination is made that the object really is a foot or something else. In the cloud a connection is made between the data and who it was that made the jump, which jump of the total amount of jumps it was and so on. Big data analyzes, and statistics are also made in the cloud. For example, place: Tampere, date: 2018, number of jumps: 450 jumps, average length of jumps: 7.68 for men and 7.05 for women and so on. The cloud is also keeping track of which users or clients, smartphones pads and so on, have requested to see the data and sends it to them.

1.-10. (canceled)

11. A method for monitoring a sports performance of an athlete comprising:
detecting by at least one movement sensor movement of the athlete;
detecting by at least one position sensor a position of the athlete;
transmitting a detection signal to a controller from at least one of the movement sensor or the position sensor;
filtering data constituting the detection signal received in the controller;
sending the filtered data to a cloud database;
storing the filtered data in the cloud database;
further filtering the filtered data using a filter in the cloud;
transmitting the further filtered data; and
displaying the further filtered data on a user interface for a user, wherein the at least one movement sensor to detect movement of the athlete is a laser scanner, and the at least one position sensor to detect position of the athlete is a 3D camera.

12. The method according to claim 11, further comprising:
further analyzing, by the controller, the detection signal and determining whether the data constituting the detection signal comprises valid information and based on the determination transmitting the data to the cloud database or discarding the data if no movement of the athlete is detected or if invalid readings of at least one of the movement sensor or the position sensor are detected.

13. The method according to claim 11, further comprising transmitting, by the controller, commands to the sensors.

14. The method according to claim 11, further comprising receiving, by the controller, control commands from the cloud for reboot, report, system status, start/stop or configuration information.

15. The method according to claim 11, further comprising transmitting, by the cloud database, data to the users, and receiving data from the users about their location, a version of the user interface and whether the user interface is functioning successfully.

16. The method according to claim 11, further comprising, displaying the data to the user in real time.

17. A system for monitoring a sports performance of an athlete comprising:
a movement sensor for monitoring movement of the athlete;
a position sensor for monitoring a position of the athlete;
a controller for receiving signals corresponding to information regarding the monitoring performed by at least one of the movement sensor or the position sensor:
the controller comprising a filter for filtering the received signals;
a cloud database for receiving and storing the data sent from the controller;
the cloud comprising a filter filtering and processing the data sent from the controller;
a user interface for displaying the processed data showing the sports performance, received from the cloud, wherein the movement sensor for monitoring movement of the athlete is a laser scanner, and the position sensor for monitoring the position of the athlete is a 3D camera.

18. The system according to claim 17, wherein the laser scanner comprises a LiDAR.

19. The system according to claim 17, wherein the 3D camera comprises an IR depth mapper.

20. The system according to claim 17, wherein the user interface comprises a smartphone, a pad, a laptop or a PC.
